(19) United States
(12) Patent Application Publication
(10) Pub. No.: US 2024/0330404 A1
(43) Pub. Date: Oct. 3, 2024

(54) SYSTEMS AND METHODS FOR PROVIDING SYNTHETIC DATA

(71) Applicant: Optum, Inc., Minnetonka, MN (US)

(72) Inventor: Mark Lefebvre, Minnetonka, MN (US)

(21) Appl. No.: 18/129,502

(22) Filed: Mar. 31, 2023

(51) Int. Cl. G06F 17/18; G16H 10/60

(52) U.S. Cl. CPC G06F 17/18; G16H 10/60

(57) ABSTRACT

Systems, methods, and apparatuses implementing a synthetic data generation system are provided herein. In some embodiments, an example synthetic data generation system may be configured to generate high-quality synthetic data that can be used for data analysis operations and/or generate one or more predictive outputs.

### BACKGROUND

Synthetic data may be used in applications where privacy concerns are relevant. Healthcare is an example of a highly regulated environment that requires derived raw data for evaluation free of site certification constraints. Healthcare data analysts may require patient data for various purposes, including statistical analysis, testing, and validating and/or training of machine learning models. However, privacy concerns including Health Insurance Portability and Accountability Act (HIPAA) restrictions may impede access to data which may lead to inadequate or insufficient amounts of data for data analysis operations. For example, a machine learning model deployed for a healthcare solution may generate inaccurate outputs due to a lack of high quality data available for training the machine learning model.

Conventional approaches may include removing Personal Identifiable Information (PII) from data sets. However, these data sets can be de-anonymized or poorly de-identified which raises significant privacy risks. In some examples, HIPAA “Safe Harbor” standards may require that certain types of PII (e.g., geographic information, date of birth, social security numbers) are removed in order for records to be considered de-identified. While de-identified data can be used to provide a somewhat representative data set, in many instances, these data sets lack sufficient detail to facilitate comprehensive data analysis and predictive data analysis. For example, fields that are needed for consumption (e.g., Current Procedural Terminology (CPT) codes, diagnosis codes, financial information, and the like) may be removed in de-identified data sets.

Therefore, systems and methods are desired that overcome challenges in the art, some of which are described above. In particular, methods and systems for generating synthetic data that can be used in various applications, including healthcare, are provided herein. Embodiments of the present disclosure can be used to provide high-quality data sets for research and data analysis in a manner that satisfies HIPAA restrictions and addresses privacy concerns.

### SUMMARY

Embodiments of the present disclosure address challenges relating to providing raw data for data analysis operations in various applications, including healthcare.

By utilizing some or all of the innovative techniques disclosed herein, various embodiments of the present invention generate realistic, useable synthetic data in a manner that conserves and optimizes the use of computational resources.

In some embodiments, a synthetic data generation system is provided. The synthetic data generation system can include at least one computing device; and a memory storing computer-readable instructions that when executed by the at least one computing device cause the at least one computing device to: receive and segment an original data set associated with a plurality of patients; process the original data set using a multivariate probabilistic distribution operation; modify at least a portion of the original data set based on an output of the multivariate probabilistic distribution operation to generate a modified data set; apply a statistical test to the original data set and the modified data set; and if a statistical test output indicates that the original data set and the modified data set are statistically different, output the modified data set as synthetic data.

In accordance with another embodiment of the present disclosure, a computer-implemented method is provided. The computer-implemented method can comprise: receiving and segmenting, by one or more processors, an original data set associated with a plurality of patients; processing, by the one or more processors, the original data set using a multivariate probabilistic distribution operation; modifying, by the one or more processors, at least a portion of the original data set based on an output of the multivariate probabilistic distribution operation to generate a modified data set; applying, by the one or more processors, a statistical test to the raw data and the modified data set; and if a statistical test output indicates that the original data set and the modified data set are statistically different, outputting, by the one or more processors, the modified data set as synthetic data.

In accordance with another embodiment of the present disclosure, a non-transitory computer-readable medium is provided. The non-transitory computer-readable medium can comprise computer-executable instructions stored thereon that when executed by at least one computing device cause the at least one computing device to: receive and segment an original data set associated with a plurality of patients; process the original data set using a multivariate probabilistic distribution operation; modify at least a portion of the original data set based on an output of the multivariate probabilistic distribution operation to generate a modified data set; apply a statistical test to the raw data and the modified data set; and if a statistical test output indicates that the original data set and the modified data set are statistically different, output the modified data set as synthetic data.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is an illustration of an exemplary system that can be used to generate synthetic data, in accordance with certain embodiments of the present disclosure;

FIG. 2 is a flowchart that illustrates an exemplary method for generating synthetic data, in accordance with certain embodiments of the present disclosure;

FIG. 3A is a schematic diagram depicting an example original data set;

FIG. 3B is a schematic diagram a schematic diagram depicting an example synthetic data set, in accordance with certain embodiments of the present disclosure; and

FIG. 4 shows an example computing environment in which example embodiments may be implemented.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

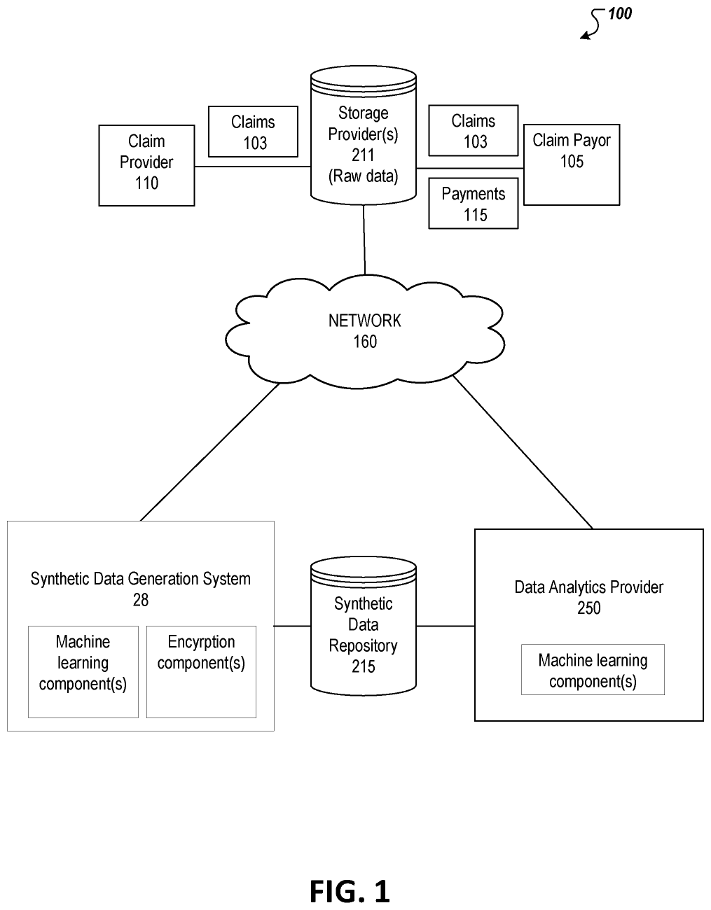

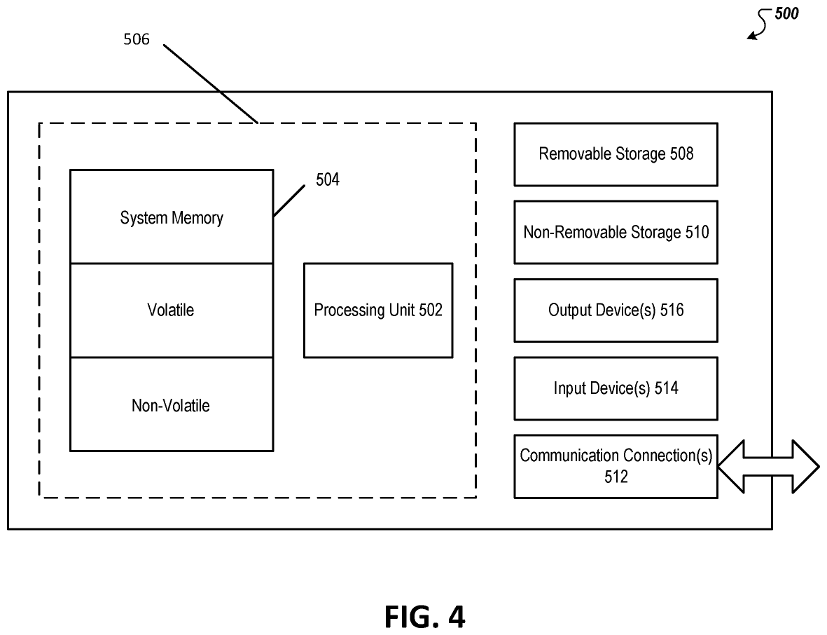

FIG. 1 is an example environment 100 implementing a synthetic data generation system 28 in accordance with certain embodiments of the present disclosure. In various embodiments, the synthetic data generation system 28 can be configured to receive raw data/an original data set from one or more entities (e.g., claim providers, claim payors, claims processors, and/or the like) and generate synthetic data that can be used for data analytics operations (e.g., testing, research, generating statistical analysis and reports). The synthetic data generation system 28 may be implemented using one or more general purpose computing devices such as the computing device 500 illustrated in FIG. 4.

As shown, the environment 100 may include one or more claim providers 110, one or more claim payors 105, a synthetic data generation system 28, one or more data analytics providers 250 (e.g., healthcare technology solutions providers), and one or more storage providers 211 in communication through a network 160. The network 160 may include a combination of private networks (e.g., LANs) and public networks (e.g., the Internet). Each of the one or more claim providers 110 and the one or more claim payors 105 may be partially implemented by one or more general purpose computing devices such as the computing device 500 illustrated in FIG. 4.

The claim provider 110 may be a medical provider or any other entity that provides claims 103 (e.g., to one or more claim payors 105). Additionally and/or alternatively, as shown, the one or may claim payors 105 may also provide claims 103. The claims 103 may be insurance claims, requests for payment for healthcare services rendered by the claim provider 110, and in some embodiments, claims 103 related to medical services provided to a patient by the claim provider 110 or another entity. In some embodiments, the claims 103 can include patient information that may be associated with a patient profile comprising member information/data, member features, and/or similar words used herein interchangeably that can be associated with a given member identifier for a patient/individual, claim(s), and/or the like. In some embodiments, a patient profile may include age, gender, known health conditions, home location, medical history, claim history, a member identifier (ID), and/or the like.

In this light, a claim provider 110 may be a physician, technician, nurse, healthcare worker, medical professional, dentist, orthodontist, optometrist, ophthalmologist, and the like. To provide for efficient storage and preserve the privacy of patients associated with the claims 103, and/or healthcare technology solutions, the environment 100 may utilize one or more storage providers 211 (e.g., dropbox storage providers (DSPs), cloud-based document storage providers, and the like). An example storage provider may store raw data (e.g., claims 103) in a database or encrypted data storage and may expose an application programming interface (API) through which the claim providers 110 and claim payors 105 may write and read documents (e.g., claims 103) from storage providers.

The claim payor 105 may include insurance companies, government entities, or any other entity that may process payments 115 and/or evaluate claims 103 on behalf of patients or other entities. In various embodiments, the data analytics provider 250 may provide administrative operations and technology solutions to claim providers 110 and/or claim payors 105. By way of example, a patient may receive medical services from a claim provider 110 (e.g., physician) that results in generation of one or more claims 103. Each claim 103 can be associated with one or more related or unrelated claim providers 110. The claim provider 110 may outsource the processing of certain aspects of the one or more claims 103 to the data analytics provider 250 or provide claims 103 that can be used for data analytics operations (e.g., to generate reports, and perform statistical analysis on operations of the claim provider 110). As depicted, the synthetic data generation system 28 can include one or more encryption components that can be used to perform de-identification operations. As further illustrated, the synthetic data generation system 28 and data analytics provider 250 can include or utilize one or more machine learning components. For example, the synthetic data generation system 28 may use one or more machine learning components to process (e.g., filter, group, analyze) raw data/data sets. In some implementations, the synthetic data generation system 28 can apply Copulas, Generative Adversarial Networks, and/or deep learning machine leaning models to generate synthetic data. The data analytics provider 250 may use one or more machine learning components to generate predictive outputs (e.g., user interface data describing outputs of various data analysis operations, reports, or the like).

In some embodiments, the synthetic data generation system 28 and/or data analytics provider 250 comprises a computer-implemented artificial intelligence-enabled engine. The term “artificial intelligence” is defined herein to include any technique that enables one or more computing devices or computing systems (i.e., a machine) to mimic human intelligence. AI includes, but is not limited to, knowledge bases, machine-learning, representation learning, and deep learning. The term “machine-learning” is defined herein to be a subset of AI that enables a machine to acquire knowledge by extracting patterns from raw data. Machine-learning techniques include, but are not limited to, logistic regression, support vector machines (SVMs), decision trees (including randomized decision forests), Naïve Bayes classifiers, AutoRegressive Integrated Moving Average (ARIMA) machine-learning algorithms, and artificial neural networks. The term “representation learning” is defined herein to be a subset of machine-learning that enables a machine to automatically discover representations needed for feature detection, prediction, or classification from raw data. Representation learning techniques include, but are not limited to, autoencoders. The term “deep learning” is defined herein to be a subset of machine-learning that enables a machine to automatically discover representations needed for feature detection, prediction, classification, etc. using layers of processing. Deep learning techniques include, but are not limited to, artificial neural network (including deep nets, long short-term memory (LSTM) recurrent neural network (RNN) architecture), or multilayer perceptron (MLP). Machine-learning models include supervised, semi-supervised, and unsupervised learning models. In a supervised learning model, the model learns a function that maps an input (also known as feature or features) to an output (also known as a target or target) during training with a labeled data set (or data set). In an unsupervised learning model, the model learns a function that maps an input (also known as feature or features) to an output during training with an unlabeled data set. In a semi-supervised model, the model learns a function that maps an input (also known as feature or features) to an output (also known as a target or target) during training with both labeled and unlabeled data.

The synthetic data generation system 28 and/or data analytics provider 250 described herein may comprise all or part of an artificial neural network (ANN). An ANN is a computing system including a plurality of interconnected neurons (e.g., also referred to as “nodes”). This disclosure contemplates that the nodes can be implemented using a computing device (e.g., a processing unit and memory as described herein), such as computing device 500 described herein. The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, in other words, the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a data set to maximize or minimize an objective function (e.g., the business goals and objectives). In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation. It should be understood that an artificial neural network is provided only as an example machine-learning model. This disclosure contemplates that the machine-learning model can be any supervised learning model, semi-supervised learning model, or unsupervised learning model. Optionally, the machine-learning model is a deep learning model. Machine-learning models are known in the art and are therefore not described in further detail herein.

A convolutional neural network (CNN) is a type of deep neural network that can be applied, for example, to non-linear workflow prediction applications, such as those described herein. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. GCNNs are CNNs that have been adapted to work on structured data sets such as graphs.

Other supervised learning models that may be utilized according to embodiments described herein include a logistic regression (LR) classifier, a Naïve Bayes' (NB) classifier, a k-NN classifier, a majority voting ensemble, and the like.

Generally, the claims 103 are electronically transmitted over the network 160 to the synthetic data generation system 28 in a standard electronic format (e.g., In the United States this may be the ANSI ASC X12N 837 format, incorporated by reference), though equivalents and other such formats are contemplated within the scope of this disclosure. The synthetic data generation system 28 can process the claims 103 and/or other data such as patient information or medical information to generate synthetic data that can be stored in a synthetic data repository 215. The data analytics provider 250 may access synthetic data stored in the synthetic data database and utilize the synthetic data in a variety of ways. By way of example, the data analytics provider 250 can generate predictive outputs relating to predicted return-on-investment (ROI), workflows, resource allocation (e.g., staffing requirements), or administrative functions that can be implemented to improve financial performance of the claim provider 110.

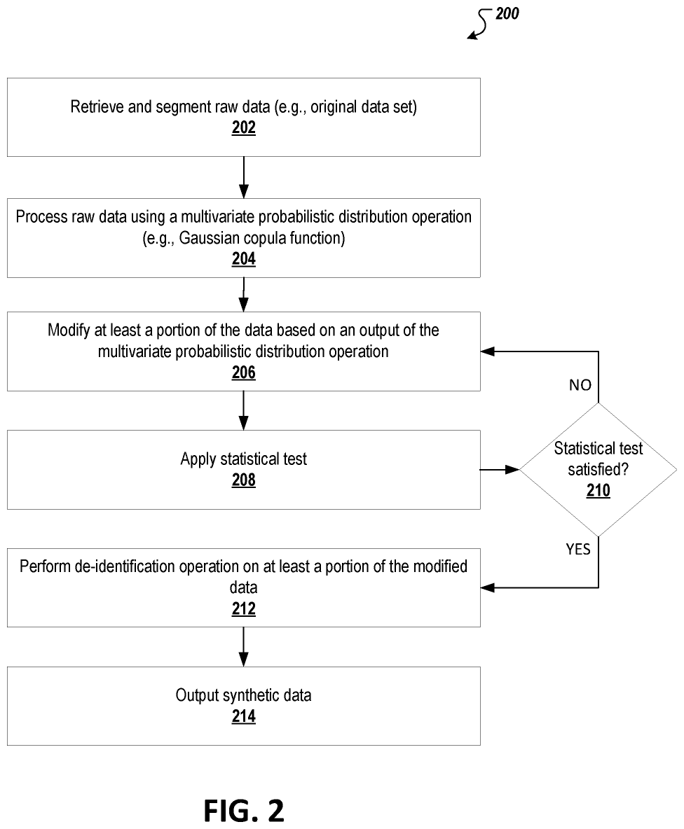

FIG. 2 is a flowchart diagram that illustrates an exemplary method 200 for providing synthetic data that can be used to generate predictive outputs.

Beginning at step/operation 202, the synthetic data generation system (such as, but not limited to, the synthetic data generation system 28 described above in connection with FIG. 1) retrieves and segments raw data (e.g., an original data set), for example, from a claim provider, claim payor, or other entity. The raw data may comprise claims, patient information, medical data, and the like. In some embodiments, step/operation 202 includes generating and/or training the synthetic data generation system (e.g., training one or more machine learning components to process the raw data). By way of example, the raw data may comprise claim information stored in a tabular form (e.g., in lines and columns) where each column is associated with a particular information field (e.g., address, patient identifier, or the like). It should be understood that each claim may comprise a plurality of columns (e.g., between 80-100 columns). In some implementations, the raw data may be in other forms such as a graph data structure. In various embodiments, the synthetic data generation system segments the original data set based on one or more parameters, for example, based on medical episodes or events as determined by CPT codes and ICD10 codes, and in accordance with user specified needs.

Subsequent to step/operation 202, the method 200 proceeds to step/operation 204. At step/operation 204, the synthetic data generation system processes the raw data using a multivariate probabilistic distribution operation, such as but not limited to, a Gaussian copula function. The synthetic data generation system can use the multivariate probabilistic distribution operation to apply a probability density function that captures a likelihood that a random sample distribution is equal to a value, x. A Gaussian copula is a multivariate distribution that models the dependence structure between random variables using a Gaussian distribution to describe correlations between the variables. By way of example only, for two random variables, x and y, a Gaussian distribution can be used to describe the relationship between x and y by specifying a value indicative of a correlation coefficient between them. A positive value may indicate that the two variables are similar, and a negative value may indicate that the two variables are dissimilar.

The multivariate probabilistic distribution operation may be used to identify patients and corresponding claims that are similar to one another or share certain characteristics, including similar medical events, episodes of care, or disease states. By way of example, the multivariate probabilistic distribution operation can be used to identify or group patients that have undergone similar medical procedures or that otherwise have similar medical histories and profiles. In some embodiments, the synthetic data generation system uses one or more trained machine learning models or components to process the raw data and find correlations between different data entities, such as a neural network, deep learning model, CNN, or the like.

Subsequent to step/operation 204, the method 200 proceeds to step/operation 206. At step/operation 206, the synthetic data generation system modifies at least a portion of the data based at least on an output of the multivariate probabilistic distribution operation. In the example of tabular data where each line is associated with a different claim, the synthetic data generation system may substitute (e.g., interchange) a first value in a field associated with a first claim with a second value in the field associated with a second claim, where the first claim is similar to the second claim based on an output generated using the multivariate probabilistic distribution operation.

Subsequent to step/operation 206, the method 200 proceeds to step/operation 208. At step/operation 208, the synthetic data generation system determines whether the raw data (e.g., original data set) and the modified data set satisfy a statistical test, such as but not limited to, a statistical hypothesis test, analysis of variance test, significance test, chi-squared test, a t-test, combinations thereof, and/or the like.

In the example of a t-test, a t-score for a first data set (original data set) and a second data set (modified data set) with an above-threshold p-value or significance level (e.g., 0.05 or greater) can indicate that the first data set and the second data set are not statistically different from one another, while a below-threshold p-value or significance level (e.g., below 0.05) can indicate that the first data set and the second data set are statistically different from one another. In some embodiments, the significance or similarity threshold can be a configurable parameter set by an end user.

At step/operation 210, if the original data set and the modified data set satisfy the statistical test, the method 200 returns to step/operation 206 and the synthetic data generation system modifies another portion of the raw data (e.g., original data set). At step/operation 210, if the original data set and the modified data set satisfy the statistical test and are statistically different from one another, then the method 200 proceeds to step/operation 212.

Optionally, at step/operation 212 the synthetic data generation system performs a de-identification operation on at least a portion of the modified data (e.g., encrypting some of the modified data using encryption component(s) depicted in FIG. 1).

Subsequent to step/operation 212, the method 200 proceeds to step/operation 214. At step/operation 214, the synthetic data generation system outputs the modified, and in some examples encrypted data set as synthetic data. For example, the synthetic data generation system can transmit the synthetic data to one or more repositories, such as, but not limited to the synthetic data repository 215 described above in connection with FIG. 1. Various entities (e.g., data analytics provider 250 described above in connection with FIG. 1) can obtain synthetic data from the one or more repositories for data analysis operations.

In some implementations, the synthetic data generation system can be configured to iteratively modify portions of the data set (e.g., modify a first portion of a data set and run a statistical test, modify a second portion of the data set and re-run the statistical test, and so on) until the modified data set is statistically different from the original data set. The synthetic data generation system may process the data in this fashion until the original data set and a modified data set are statistically different. By modifying the data set in an iterative fashion, the synthetic data generation system ensures that the synthetic data being generated is realistic enough to be usable for data analysis and for generating predictive outputs while conserving computational resources. Experimental results demonstrate that synthetic data generated in accordance with embodiments of the present disclosure satisfies various statistical tests and is less vulnerable to de-identification. In particular, t-tests were conducted to test continuous data (e.g., dollar amounts) and Chi Square Tests were conducted to test categorical and count variables to demonstrate that the generated synthetic data is statistically distinct from the original data set. In contrast with embodiments of the present disclosure, conventional techniques may randomly scramble data in an original data set, but such techniques may output nonsensical and non-cohesive data that cannot be subsequently analyzed in a meaningful way. For example, using a conventional random technique, data associated with a patient that has undergone an x-ray may be partially interchanged with data associated with a patient that has undergone chemotherapy. In this example, because the patient that has undergone an x-ray and the patient that has undergone chemotherapy are dissimilar, the modified data that is generated is unrealistic and if used for data analytics operations, would result in inaccurate predictive outputs and/or analysis.

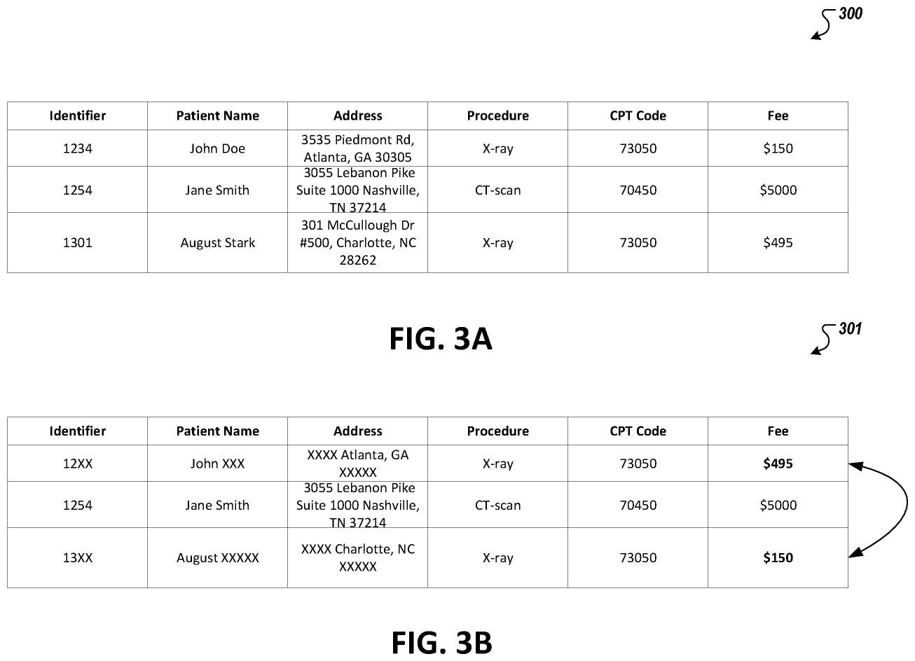

Referring now to FIG. 3A, a schematic diagram depicting an example original data set 300 (e.g., raw data) is provided. As illustrated, the original data set 300 comprises claim information for a plurality of patients presented in tabular form. The original data set 300 includes PII fields such as an identifier, patient name, and address. Additionally, the original data set 300 includes non-PII fields such as procedure type, CPT code, and fee information. The synthetic data generation system can determine, for example using the above-noted segmentation operation and/or multivariate probabilistic distribution operation (e.g., Gaussian copula) that a first data entity associated with identifier 1234 and a second data entity associated with 1301 are similar to one another and/or share one or more characteristics (e.g., common medical events or episodes).

Referring now to FIG. 3B, a schematic diagram depicting an example synthetic data set 301 is provided. As shown, the synthetic data set 301 comprises claim information for a the same plurality of patients in the original data set 300 described above. Similarly, the synthetic data set 301 includes PII fields such as an identifier, patient name, and address. As shown in FIG. 3B, subsequent to determining that the first data entity associated with identifier 1234 and the second data entity associated with identifier 1301 are similar to one another, the synthetic data generation system modifies at least a portion of the data in the original data set 300 presented in FIG. 3A. As shown in FIG. 3B, the CPT codes for the first data entity associated with identifier 1234 in the first row and the third data entity associated with identifier 1301 are identical/correspond with one another. Accordingly, the values stored in the respective fee information fields have been interchanged. As further depicted in FIG. 3B, in the synthetic data set 301, the information in the PII fields has been encrypted (e.g., using a de-identification operation) to obscure the PII related information. In particular, the identifier, patient name, and address fields have been modified to obscure personal information.

FIG. 4 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, cloud-based systems, microprocessor-based systems, network personal computers (PCs), minicomputers, mainframe computers, embedded systems, distributed computing environments that include any of the above systems or devices, and the like. The computing environment may include a cloud-based computing environment.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 4, an example system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random-access memory (RAM)), non-volatile (such as read-only memory (ROM), flash memory, etc.), or some combination of the two. This most basic configuration is illustrated in FIG. 4 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 4 by removable storage 508 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A synthetic data generation system comprising:
at least one computing device; and
a memory storing computer-readable instructions that when executed by the at least one computing device cause the at least one computing device to:
receive and segment an original data set associated with a plurality of patients;
process the original data set using a multivariate probabilistic distribution operation;
modify at least a portion of the original data set based on an output of the multivariate probabilistic distribution operation to generate a modified data set;
apply a statistical test to the original data set and the modified data set; and
if a statistical test output indicates that the original data set and the modified data set are statistically different,
output the modified data set as synthetic data.

2. The synthetic data generation system of claim 1, wherein the at least one computing device further comprises at least one encryption component that is configured to perform a de-identification operation on at least a portion of the modified data set.

3. The synthetic data generation system of claim 1, wherein the at least one computing device is further configured to generate the modified data set by:
iteratively modifying different portions of the original data set and re-applying the statistical test until the statistical test output indicates that the original data set and the modified data set are statistically different.

4. The synthetic data generation system of claim 1, wherein the multivariate probabilistic distribution operation comprises a Gaussian copula function.

5. The synthetic data generation system of claim 1, wherein the synthetic data is used to generate one or more predictive outputs.

6. The synthetic data generation system of claim 1, wherein the at least one computing device further comprises at least one machine learning component.

7. The synthetic data generation system of claim 5, wherein the at least one machine learning component comprises a neural network or a convolutional neural network.

8. The synthetic data generation system of claim 1, wherein the raw data comprises at least one of claim information and medical information.

9. A computer-implemented method comprising:
receiving and segmenting, by one or more processors, an original data set associated with a plurality of patients;
processing, by the one or more processors, the original data set using a multivariate probabilistic distribution operation;
modifying, by the one or more processors, at least a portion of the original data set based on an output of the multivariate probabilistic distribution operation to generate a modified data set;
applying, by the one or more processors, a statistical test to the raw data and the modified data set; and
if a statistical test output indicates that the original data set and the modified data set are statistically different, outputting, by the one or more processors, the modified data set as synthetic data.

10. The computer-implemented method of claim 9, further comprising:
performing, by the one or more processors, a de-identification operation on at least a portion of the modified data set.

11. The computer-implemented method of claim 9, wherein generating the modified data set comprises:
iteratively modifying, by the one or more processors, different portions of the original data set and re-applying the statistical test until the statistical test output indicates that the original data set and the modified data set are statistically different.

12. The computer-implemented method of claim 9, wherein the multivariate probabilistic distribution operation comprises a Gaussian copula function.

13. The computer-implemented method of claim 9, wherein the synthetic data is used to generate one or more predictive outputs.

14. The computer-implemented method of claim 9, wherein processing the original data set using a multivariate probabilistic distribution operation comprises using at least one machine learning model component.

15. The computer-implemented method of claim 13, wherein the at least one machine learning component comprises a neural network or a convolutional neural network.

16. The computer-implemented method of claim 9, wherein the original data set comprises at least one of claim information and medical information.

17. A non-transitory computer-readable medium with computer-executable instructions stored thereon that when executed by at least one computing device cause the at least one computing device to:
receive and segment an original data set associated with a plurality of patients;
process the original data set using a multivariate probabilistic distribution operation;
modify at least a portion of the original data set based on an output of the multivariate probabilistic distribution operation to generate a modified data set;
apply a statistical test to the raw data and the modified data set; and
if a statistical test output indicates that the original data set and the modified data set are statistically different, output the modified data set as synthetic data.

18. The non-transitory computer-readable medium of claim 17, wherein outputting the synthetic data comprises transmitting the synthetic data to one or more data repositories.

19. The non-transitory computer-readable medium of claim 17, wherein the computer-executable instructions further comprise instructions to cause the at least one computing device to:
perform a de-identification operation on at least a portion of the modified data set.

20. The non-transitory computer-readable medium of claim 17, wherein generating the modified data set comprises:
iteratively modifying different portions of the original data set and re-apply the statistical test until the statistical test output indicates that the original data set and the modified data set are statistically different.
